(19) United States
(12) Patent Application Publication
(10) Pub. No.: US 2009/0234268 A1
(43) Pub. Date: Sep. 17, 2009

(54) TAMPON PLEDGETS WITH IMPROVED LEAKAGE PROTECTION

(75) Inventors: Robert T. Jorgensen, JR., Middletown, DE (US); Yinka Abdul, Newark, DE (US); Philip Burkhardt, Magnolia, DE (US)

Correspondence Address: MICHAUD-DUFFY GROUP LLP, 306 INDUSTRIAL PARK ROAD, SUITE 206, MIDDLETOWN, CT, 06457, US

(73) Assignee: PLAYTEX PRODUCTS, LLC, Shelton, CT (US)

(21) Appl. No.: 12/400,021

(22) Filed: Mar. 9, 2009

Related U.S. Application Data
(60) Provisional application No. 61/035,606, filed on Mar. 11, 2008.

(51) Int. Cl. A61F 13/20

(52) U.S. Cl. 604/15; 604/385.17

(57) ABSTRACT

An increased absorbency tampon pledget with an absorbent capacity of 15-18 grams and an increased dry bulk density has a density of at least 0.20 g/cc and can be housed in a smaller applicator than typically provided with commercially available increased absorbency applicator tampons. The increased dry bulk density provides better leakage protection, placement control, and user comfort.

### CROSS REFERENCE TO RELATED APPLICATION

This application claims the benefits of U.S. Provisional Patent Application Ser. No. 61/035,606, filed Mar. 11, 2008, entitled “Tampon Pledgets With Improved Leakage Protection,” the content of which is incorporated herein by reference in its entirety.

### FIELD OF THE INVENTION

The present invention relates generally to an improved tampon pledget. More particularly, the present invention relates to an improved tampon pledget having a higher dry bulk density and smaller size, as compared to similar absorbent capacity tampon pledgets. The increased dry bulk density provides better leakage protection, placement control and user comfort.

### BACKGROUND

Currently, tampon users have a choice of five FDA controlled product capacities: Lites (“light duty” or “light absorbency”)<6 grams, Regular 6-9 grams (“regular absorbency”), Super 9-12 grams, Super Plus 12-15 grams (both of which are “high absorbency”), and Ultra 15-18 grams (“increased absorbency”). These tampons may have an applicator (such as cardboard or plastic) or may be inserted digitally. Despite the wide variety of tampon pledgets available, they continue to have a propensity to leak prematurely. This is due to improper placement within the vaginal canal and slow pledget expansion upon contact with fluid. And, while the range of absorbent capacities of tampon pledgets covers capacity for menses flow of many women, a need for other sizes exists.

Many users are in need of the increased absorbency tampon pledgets. The increased absorbency tampon pledgets, however, generally have a greater mass, which results in a larger applicator size and higher ejection forces. These characteristics may cause a user to choose a lower absorbent capacity tampon, yet still be in need of a higher absorbent capacity.

A pre-expanded tampon with a lower overall density and a compressed tampon with higher expansion forces were developed to provide tampons with quick expansion that rapidly fill the vaginal cavity. Despite advances in the field, however, the need for improved performance requirements persists, specifically with respect to absorbent capacity, bypass leakage protection, comfort, placement control and ease of use.

### SUMMARY OF THE INVENTION

The improved pledget of the present invention has a higher dry bulk density and smaller size, as compared to similar absorbent capacity tampon pledgets. The increased dry bulk density provides better leakage protection, placement control and user comfort.

In one aspect, the present invention resides in an increased absorbency tampon pledget with an absorbent capacity of 15-18 grams and an increased dry bulk density. This increased absorbency tampon pledget has a density of at least 0.20 g/cc, and more preferably in the range of about 0.20 g/cc to about 0.80 g/cc, and can be housed in a smaller applicator than typically provided with commercially available increased absorbency applicator tampons. In an alternative embodiment, an increased absorbency digital tampon pledget with an absorbent capacity of 15-18 grams, and at least ⅔ of the pledget's length having a density of at least 0.36 g/cc, is provided.

In another embodiment of the present invention a tampon pledget including an applicator, has a plunge pledget force in the range of about 0.050 N/mm to about 0.500 N/mm, and more preferably in the range of about 0.050 N/mm to about 0.300 N/mm.

In a further embodiment of the present invention a tampon pledget applicator assembly has an applicator ejection force that provides for easy ejection of the pledget from the applicator.

In a further embodiment of the present invention a tampon pledget (applicator or digital) with an expansion delta from t=0 minutes (dry) to t=12 minutes (wet) of 45% or greater, and more preferably 50% or greater, is provided.

In yet a further embodiment of the present invention, a tampon pledget has a radial expansion width at t=0 minutes in the range of about 12 mm to about 25 mm, and more preferably in the range of about 12 mm to about 20 mm. In another embodiment of the present invention, an applicator tampon pledget has a radial expansion width at t=12 minutes in the range of about 22 mm or greater, and more preferably in the range of about 25 mm to about 40 mm. In an alternative embodiment, a digital tampon pledget where at least ⅔ of the length of the pledget has a radial expansion width at t=12 minutes of about 26 mm or greater, and more preferably about 28 mm or greater.

In still a further embodiment of the present invention, an applicator tampon pledget has an expansion rate at t=12 minutes of about 0.60 mm/minute or greater, and more preferably of about 0.65 mm or greater. In an alternative embodiment, a digital tampon pledget wherein at least ⅔ of the length of the pledget has an expansion rate of 0.65 mm/minute is provided.

In still a further embodiment of the present invention, an increased absorbency applicator tampon having an absorbent capacity of 15-18 grams, and further comprising a radial expansion width at t=4 minutes in the range of about 18 mm to about 35 mm, and more preferably in the range of about 19 mm to about 30 mm, and a radial expansion width at t=7 minutes in the range of about 20 mm to about 35 mm, and more preferably in the range of about 22 mm to about 33 mm is provided. In an alternative embodiment, an increased absorbency digital tampon having an absorbent capacity of 15-18 grams, wherein at least ⅔ of the length of the pledget has a radial expansion at t=4 minutes of 25 mm or greater, and more preferably about 27 mm or greater, and wherein at least ⅔ of the length of the pledget has a radial expansion at t=7 minutes of 25 mm or greater, and more preferably about 27 mm or greater is provided.

### BRIEF DESCRIPTION

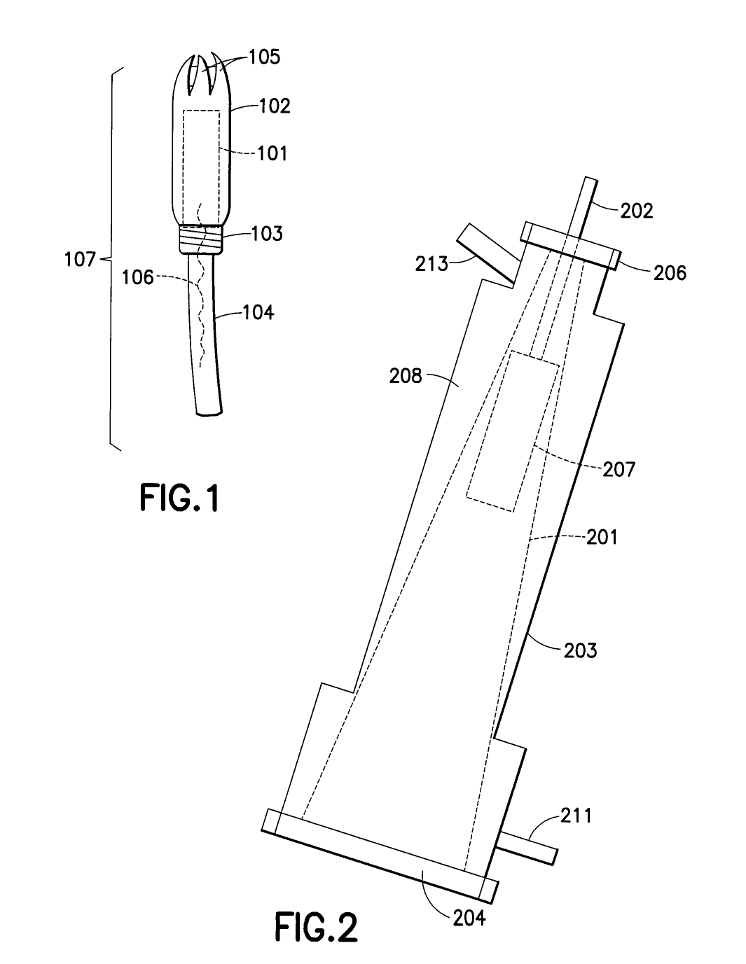

FIG. 1 is a front view of an applicator type tampon of the present invention;

FIG. 2 is a front view of the set up for the absorbent capacity test method;

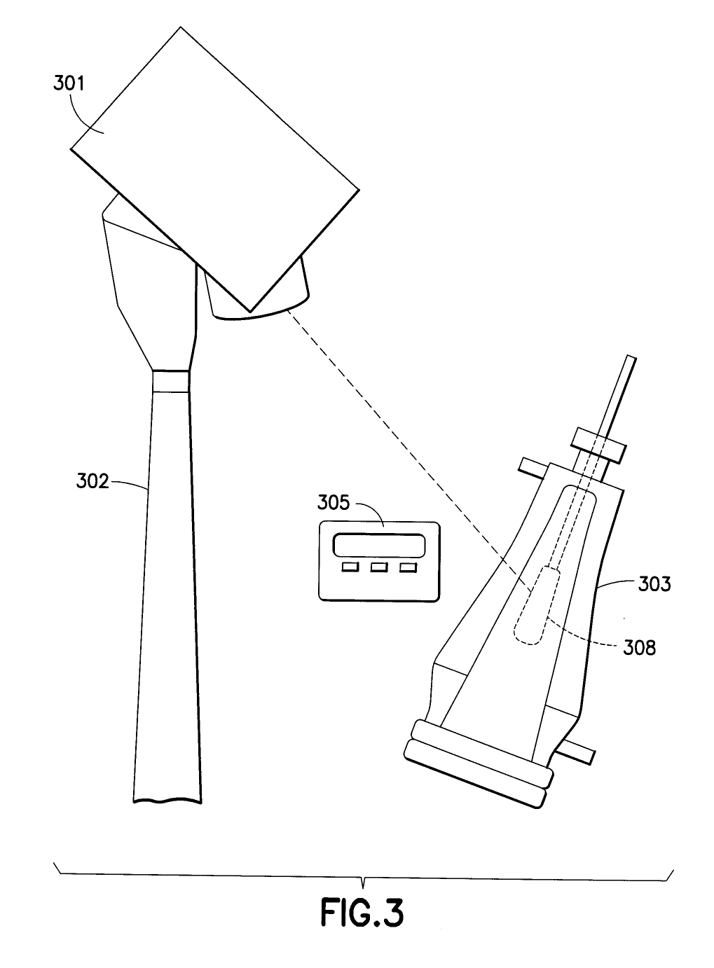

FIG. 3 is a front view of the set up for the radial expansion width and expansion rate test methods;

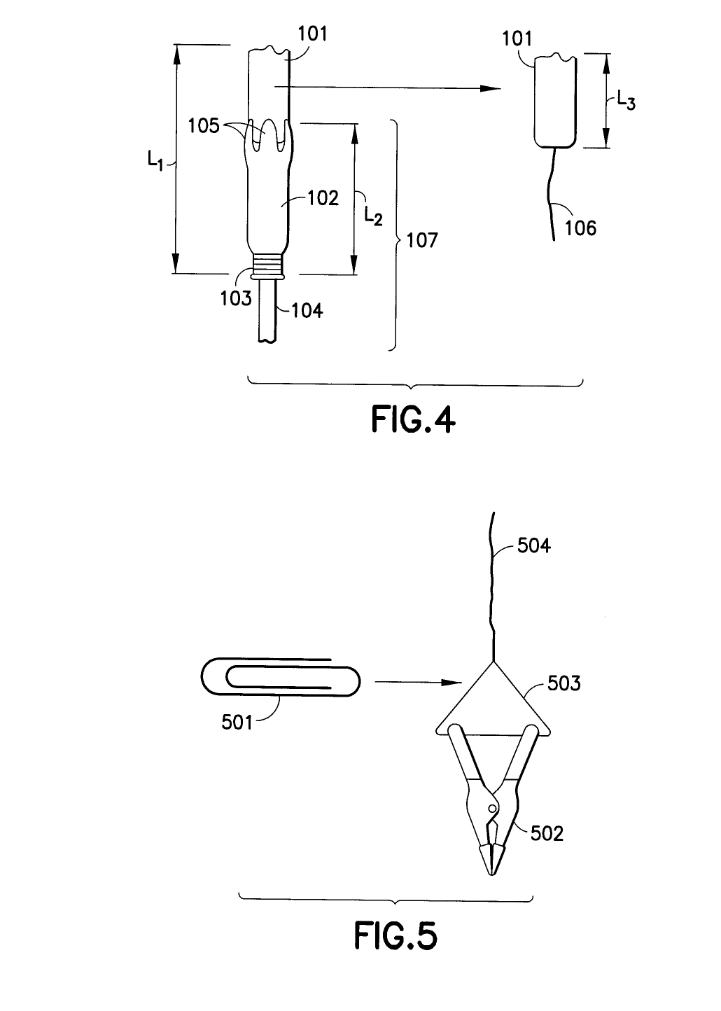

FIG. 4 is a front view of an applicator type tampon with an embedded pledget; and

FIG. 5 is a front view of the paper clip, mini-spring clip and fishing line assembly.

### DETAILED DESCRIPTION

As used herein, the terms “tampon,” “pledget,” and “tampon pledget” are intended to be used interchangeably.

Both in-vivo and in-vitro testing demonstrates that commercially available tampon pledgets do not protect well against bypass leakage. In-vivo testing shows that the typical user places the tampon too far within the vaginal canal. This deep placement is not optimal to absorb fluid. And, in-vitro tests show that pledget expansion is not optimal. The results of the in-vivo and in-vitro testing indicate that there is a propensity for tampon pledgets to leak prematurely.

As used herein, the term “tampon,” refers to any type of compressed absorbent structure, which is fluid expanding, and that can be inserted into the vaginal canal, with or without an applicator, for the absorption of fluid therefrom. FIG. 1 illustrates the front view of an applicator type tampon. The tampon pledget 101 is housed within the barrel 102 of the applicator 107. A finger grip area 103 is located at the base of the barrel 102. A plunger 104 moveably engages with the barrel 102 through the finger grip area 103. A string 106 may be connected to the tampon pledget 101 for removal from the vaginal cavity. The top of the barrel 101 has several petals 105 forming an opening for ejection of the pledget 101.

The tampon pledget 101 of the present invention has an increased dry density profile, which provides higher absorbent capacity in a smaller size, as compared to commercially available tampons with similar absorbent capacities. The increased density profile also results in increased radial expansion width, rate of expansion, and expansion delta, and improved applicator ejection and plunge pledget forces. These improved measurable quantities of the present invention translate to improved end use tampon qualities, such as higher absorbent capacity, easier insertion, smaller applicator 107, easier ejection from the applicator 107, reduction of bypass leakage, quick and significant expansion in the presence of fluid, and improved user placement control.

The tampon pledget's measurable quantities include:

Absorbent Capacity: Currently, tampon users have a choice of five FDA controlled product absorbent capacities: light absorbency<6 grams, regular absorbency 6-9 grams, Super 9-12 grams, Super Plus 12-15 grams (both of which are higher absorbency than the regular absorbency), and increased absorbency 15-18 grams. The guidelines regarding standard FDA Syngyna capacity are outlined in the Federal Register Part 801, §801.43 and testing was performed in accordance with U.S. Pat. Nos. 6,837,882 and 6,554,814 and U.S. Patent Application Publication No. 2006/0235362 (all of which are herein incorporated by reference in their entireties).

Dry Bulk Density: The density of the tampon is measured at specific points along the longitudinal length of the tampon prior to exposure to fluid. The greater density results, in a smaller size that contributes, inter alia, to lower forces needed to eject the tampon pledget from its applicator. This in turn, leads to greater ease of insertion. Moreover, the increased density also helps to increase expansion rate once exposed to fluid.

Tampon Ejection Force: The amount of force needed to eject a tampon pledget from an applicator. The ejection force is measured in ounces. The ejection force is determined in part by the size, design and type of material used for the applicator and tampon pledget. It is desirable to have an applicator and pledget assembly that provides for easy ejection of the pledget from the applicator.

Plunge Pledget Force: This is the amount of force needed to remove the tampon pledget from an applicator while the tampon still remains partially within the applicator's petals. The portion of pledget that is retained within the applicator's petals is referred to as the embedded portion. The applicator's petals retention of at least a portion of the tampon pledget ensures that a user is able to place the tampon pledget according to preference within the vaginal cavity, which translates, for example, to comfort, ease of use and ease of insertion. The plunge pledget force is measured in N/mm.

Radial Expansion Width and Expansion Rate: Faster expanding tampons cover the vaginal cavity quickly, reducing the potential for bypass leakage. The width of the tampon is measured at specific time intervals (t) of 0, 4, 7 and 12 minutes, for example, at the top, bottom, and widest radial distances. The width is measured in mm. When t=0 minutes, the tampon pledget is dry and has not yet contacted fluid. At t=12 minutes, the tampon is wet and has been in contact with fluid for 12 minutes. It should be appreciated by those of skill in the art, that any time intervals may be used. For example, more frequent time intervals over a shorter time period may be used when evaluating pledgets of lower absorbent capacity. The tampon pledget of the present invention has a narrower diameter at t=0 minutes than commercially available tampon pledgets of the same absorbent capacity. This alleviates a potential tampon user's apprehension of a larger sized tampon and provides an acceptable insertion comfort. The expansion rate is measured in mm/minute and indicates the time it takes for the diameter of the widest part of the pledget to reach a certain width. This determines the amount of time it takes for the pledget to expand and cover a significant portion of the vaginal cavity. A larger radial width and quicker expansion rate translates to less bypass leakage.

Expansion Delta: This term relates radial expansion width to expansion rate, meaning the change in the pledget dimensions over time. A high expansion delta accounts for a tampon pledget that blooms to a high degree during the tampon pledget's initial 12 minutes, for example, of exposure to fluid. A tampon pledget of this nature is comparatively small at t=0 minutes and blooms quickly to cover the vaginal cavity to prevent bypass leakage, showing a high degree of change over time. The formula for calculating the expansion delta percentage is as follows:

$$Expansion Delta = \frac{\begin{matrix}( Expansion Width @ t = 12 ) - \\ ( Expansion Width @ t = 0 )\end{matrix}}{( Expansion Width @ t = 0 )} * 100$$

In one embodiment of the present invention, the tampon pledget is a compressed large mass tampon that has a smaller initial size, as compared to similar absorbent capacity tampons available in the art, while still being able to quickly expand covering the vaginal cavity as fluid is introduced, providing bypass leakage protection. Decreasing pledget size, while increasing bypass leakage protection via a rapidly expanding pledget, offers security and comfort to a user.

In another embodiment of the present invention an increased absorbency tampon pledget has an applicator ejection force that a user can easily manage. This requires an applicator and pledget assembly design such that the pledget can easily eject from the applicator.

In another embodiment of the present invention an increased absorbency applicator tampon pledget with an absorbent capacity of 15-18 grams and an increased dry bulk density is provided. This increased absorbency tampon pledget has a density of at least 0.20 g/cc, and more preferably in the range of about 0.20 g/cc to about 0.80 g/cc, and can be housed in a smaller applicator than typically provided with commercially available increased absorbency applicator tampons. In an alternative embodiment, an increased absorbency digital tampon pledget with an absorbent capacity of 15-18 grams, and at least ⅔ of the pledget's length having a density of at least 0.36 g/cc, is provided. The increased density results in the absorbent capacity of an increased absorbency tampon, while maintaining a smaller overall size, thus decreasing user apprehension and discomfort, and making insertion easier.

In another embodiment of the present invention a tampon pledget including an applicator, has a plunge pledget force in the range of about 0.050 N/mm to about 0.500 N/mm, and more preferably in the range of about 0.050 N/mm to about 0.300 N/mm. This force allows the user placement control by the applicator retaining enough of the tampon pledget so the user can guide the pledget into a preferred placement. This is measured by the plunge pledget forces and embedded portion of the tampon pledget.

In a further embodiment of the present invention a tampon pledget (applicator or digital) with an expansion delta from t=0 minutes (dry) to t=12 minutes (wet) of 45% or greater, and more preferably 50% or greater, is provided. These expansion changes coincide with ease of insertion from the comparatively smaller initial size when compared to other similarly sized tampon pledgets. Bypass leakage is likewise improved due to a quick expansion to cover the vaginal cavity as fluid is introduced.

In yet a further embodiment of the present invention, a tampon pledget has a radial expansion width at t=0 minutes in the range of about 12 mm to about 25 mm, and more preferably in the range of about 12 mm to about 20 mm. In another embodiment of the present invention, an applicator tampon pledget has a radial expansion width at t=12 minutes in the range of about 22 mm or greater, and more preferably in the range of about 25 mm to about 40 mm. In an alternative embodiment, a digital tampon pledget where at least ⅔ of the length of the pledget has a radial expansion width at t=12 minutes of about 26 mm or greater, and more preferably about 28 mm or greater is provided. In still a further embodiment of the present invention, an applicator tampon pledget has an expansion rate at t=12 minutes of about 0.60 mm/minute or greater, and more preferably of about 0.65 mm or greater. In an alternative embodiment, a digital tampon pledget wherein at least ⅔ of the length of the pledget has an expansion rate of 0.65 mm/minute is provided. These embodiments offer a pledget that rapidly expands to a width that significantly covers the vaginal cavity, therefore preventing bypass leakage. The quick expansion also lends itself to improved comfort during wear, as the compressed material quickly softens upon expansion.

In still a further embodiment of the present invention, an increased absorbency applicator tampon having an absorbent capacity of 15-18 grams, and further comprising a radial expansion width at t=4 minutes in the range of about 18 mm to about 35 mm, and more preferably in the range of about 19 mm to about 30 mm, and a radial expansion width at t=7 minutes in the range of about 20 mm to about 35 mm, and more preferably in the range of about 22 mm to about 33 mm is provided. In an alternative embodiment, an increased absorbency digital tampon having an absorbent capacity of 15-18 grams, wherein at least ⅔ of the length of the pledget has a radial expansion at t=4 minutes of 25 mm or greater, and more preferably about 27 mm or greater, and wherein at least ⅔ of the length of the pledget has a radial expansion at t=7 minutes of 25 mm or greater, and more preferably about 27 mm or greater is provided. These embodiments illustrate, for example, a pledget that is continually expanding in the presence of fluid.

### EXAMPLES

### Test Methods

Standard Syngyna Test (Absorbent Capacity): Testing was done in accordance with Standard FDA Syngyna capacity as outlined in the Federal Register Part 801, §801.43, as illustrated in FIG. 2. An un-lubricated condom 201, with tensile strength between 17-30 Mega Pascals was attached to the large end of a glass chamber 203 with a rubber band 204 and pushed through until all slack was removed. The tip of the condom 209 was stretched over the end of the chamber 203 and secured with another rubber band 206. A tampon 207 pre-weighed (to the nearest 0.01 gram) was placed within the condom membrane 201 so that the center of gravity of the tampon 207 was at the center of the chamber 203 within the condom membrane 201. An infusion needle (14 gauge) 202 was inserted through the septum created by the condom tip 201 until it contacted the end of the tampon 207. The inner chamber 208 was filled with water pumped from a temperature controlled water bath through an inlet 211 and out an outlet 213 to maintain the average temperature of 27±° C. The water was returned to the water bath.

The Syngyna fluid (10 grams sodium chloride, 0.5 grams Certified Reagent Acid Fuchsin, diluted to 1,000 milliliters with distilled water) was then pumped through the infusion needle 202 at a rate of 50 milliliters per hour. The test was terminated when the tampon 207 was saturated and the first drop of fluid exited the apparatus. The test was aborted if fluid was detected in the folds of the condom before the tampon 207 was saturated. The water was then drained from chamber 208 via the inlet 211, and the tampon 207 was removed and immediately weighed to the nearest 0.01 grams. The absorbent capacity of the tampon 207 was determined by subtracting its dry weight from the wet final weight. The condom 201 was replaced after 10 tests or at the end of the day during which the condom 201 was used in testing, whichever occurred first.

Density Testing: With Vernier Calipers, the diameter and length of the tampon 101 ejected from applicator 107 was measured. Using several sample pledgets 101 of the same lot number, the moisture content was determined by utilizing a moisture analyzer (Mettler Toledo HR73 Halogen Moisture Analyzer). The remaining tampons 101 were weighed to the nearest 0.01 grams, correcting for moisture content. First the total pledget 101 volume was measured by pouring approximately 1 cc of lab salt (sodium chloride crystals, reagent grade, obtained from VWR catalog number VWGY30-5, Lot#41044109) into the bottom of a calibrated graduated cylinder (Kimble Kimax 50 ml; ±0.4 ml). The whole pledget 101 with the string 106 removed was placed in the graduated cylinder on top of the 1 cc of lab salt. Then the remaining 9 cc of lab salt was poured over the pledget 101. The graduated cylinder was tapped several times until the displacement reading was stable. The displacement reading from the graduated cylinder was recorded. Then the pledget 101 was removed and all excess salt was removed. Utilizing the “EdgeCraft” 662 Electric Slicer and Holder, each tampon 101 was sliced into 0.25 inch (6.35 mm) segment series. The above displacement procedure was repeated for each of the segment series. The pledget 101 density was then calculated using the following formula:

$$Pledget Density = \frac{Segmented Pledget Weight}{( displacement - 10 )}$$

Radial Expansion Width and Expansion Rate: The Standard FDA Syngyna capacity test as stated above according to the Federal Register Part 801, §801.43 was modified as illustrated in FIG. 3. An Olympus E510 Digital SLR or Nikon D50 camera 301 was attached with a Pro-Master 58 mm 1× Marco filter to a 58 mm Olympus lens. The camera 301 automatically focused and flashed when the picture was taken. The camera 301 was mounted on a tripod 302 at a 30-degree angle (60-degrees by protractor) parallel to the syngyna chamber 303. The focal point was the center mid point on a small calibrated rule (not shown) inside the syngyna chamber 303 and a timer 305 was placed alongside the syngyna chamber 303. Both were viewable through the camera 301 lens while keeping the camera 301 as close as possible. Light was provided by two black lights 306, 307 (not shown) mounted alongside and facing the syngyna chamber 303. Photographic contrast control by pivoting the black lights 306, 307 until the desired contrast was achieved. The first picture taken was of the tampon 308 in the syngyna chamber 303 and this was marked as t=0 minutes (dry). The syngyna fluid was changed to 10 grams sodium chloride, 0.5 grams Certified Reagent Acid Fuchsin, 3.50 g of Bonn Trace dye (a yellow/green fluorescent dye from Bonneau Dye Corporation, 10815 Briggs Road, Cleveland, Ohio 44111) diluted to 1,000 milliliters with distilled water. Photographs were taken at 1 minute intervals. The photographs were analyzed using Scion Inage analysis software. Three measurements were recorded: (1) the top—approximately 5 mm from the upper most end of the tampon 308, (2) the bottom—approximately 7 mm from the bottom most edge of the tampon 308, and (3) several measurements were made to determine the widest radial diameter. Calibration measurements can be made on known cylinders.

$$Expansion Rate ( mm \text{/} minute ) = \frac{( ending width - starting width )}{Time}$$

Plunge Length: Samples are acclimated to standard room temperature conditions (22±1° C. and 50% humidity). The tampon 101 removal string 106 was cut at the base of the plunger 104. Gripping the applicator barrel 102, with the tampon 101 still fully within the applicator 102 was set in a vertical position on a scale. A slow continuous downward force was applied until 44 oz. was reached. If the pledget 101 was fully expelled, the recorded embedded length was marked “O, NA.” If the pledget 101 remained within the applicator 107, the pledget 101 was referred to as embedded as illustrated in FIG. 5, such that a portion of the tampon pledget 101 remained within the barrel 102 and was engaged with the petals 105 of the applicator 107, after being ejected via the plunger 104. The embedded tampon pledget 105 while still engaged with the applicator 107, was placed on the Linear Encorder (Ono Sokki Co. LTD) for measurement. The linear encoder was set up using 2″ and 1″ Starret Gage Blocks (Distributor Webber Gage Div., The LS Starret Com., 2400 Detroit Road, Cleveland, Ohio 44145). The length from the applicator's barrel 102 bottom to the top of the embedded pledget (101) was measured and recorded as L1. The next measurement taken was the length from the applicator barrel 102 bottom to each of the petals 105 tops using Starret Digital Calipers (Model #721A). This measurement was recorded as L2. The tampon pledget 101 was removed from the applicator 107 and the length was measured on a linear encoder using the Starret block Gages consisting of the 1″ block. This measurement was recorded as L3. The embedded length was calculated using the following formula:

Embedded Length=(L2+L3)−L1

Pledget Plunge Force: Gripping the applicator barrel 102, the applicator 107 with tampon 101 was set in a vertical position on a scale. A slow continuous downward force was applied until 44 oz is reached. If the pledget was fully expelled the sample was disregarded. If the pledget 101 remained within the applicator 107, the pledget 101 was referred to as embedded as illustrated in FIG. 5, such that a portion of the tampon pledget 101 remained within the barrel 102 and was engaged with the petals 105 of the applicator 107, after being ejected via the plunger 104. The embedded tampon pledget 101 while still engaged within the applicator 107 at the petals 105 was used in the second step. FIG. 4 illustrates a paper clip 503, mini-spring clip 502 and fishing line 504 assembly used in this test method. Two GEM paper clips 501 (No. 1 GEM clip, model XP40020) were unfolded to form a straight line. Each straightened paperclip was strung with a mini-spring clamp 502 (2¾″ coated plastic tip mini-spring clips, model #614-593 “WorkForce” Atlanta, Ga. from Home Depot SKU# 35794626228). Then each paper clip 501 was bent into the shape of a closed triangle 503. A 12″ piece of the fishing line 504 (0.0198″ dia., 25 lb. Test Shakespeare model #W550 or equivalent) was secured to the top of the twisted triangular paper clip. One clamp 502/paper clip 503/fishing line 504 assembly was secured to the upper pneumatic test grips of an Instron unit (model #4411 with standard, with load cell model #55843). The second assembly was secured to the lower pneumatic test grips. The tampon string 106 was cut off at the finger grip 103 bottom. The plunged/partly ejected tampon 101 applicator 107 assembly was placed vertically into the upper and lower mini-spring clamps 502. The upper utility clip 503 was attached to the top of the fully plunged partially embedded tampon 101, while making sure that the fishing line 504 was reasonably taught. An Instron tensile strength test was conducted and the data was recorded.

Tampon Ejection Force: Gripping the applicator barrel 102, the applicator 107 with tampon 101 was set in a vertical position on a scale. A slow continuous downward force was applied until 44 oz is reached. The highest scale reading was then recorded and reported as the Tampon Ejection Force.

Data from an in-vitro comparison using the above test methods, of a high dry density increased absorbency tampon pledget of the present invention and an OB® Digital Ultra Tampon, is illustrated in Tables 1 and 2. As Table 1 shows, the absorbent capacity for increased absorbency tampon pledgets of the present invention (column A), as well as commercially available pledgets (column B), are within the 15-18 gram range.

TABLE 1
Pledget Comparisons

| | A | B |
|---|---|---|
| Absorbent | | |
| Capacity | grams | Grams |
| | 17.1 ± 0.5 | 16.7 ± 1.0 |
| Density | (g/cc) | (g/cc) |
| String End | 0.25 ± 0.04 | 0.33 ± 0.03 |
| Center | 0.61 ± 0.11 | 0.36 ± 0.02 |
| Petal End | 0.58 ± 0.13 | 0.34 ± 0.03 |
| Expansion Rate @ | | |
| 12 min. | (mm/min) | (mm/min) |
| Top | 1.03 | 0.63 |
| Bottom | 0.72 | 0.60 |
| Widest | 1.27 | 0.82 |
| Plunge Force | N/mm | N/mm |
| | 0.101 | 0 (no applicator) |

TABLE 2
Radial Expansion Width and Expansion Delta Comparison

| Time | Top (mm) | | Bottom (mm) | | Widest (mm) | |
|---|---|---|---|---|---|---|
| (minutes) | A | B | A | B | A | B |
| 0 | 15.0 ± 2.0 | 18.4 ± 2.5 | 17.0 ± 2.1 | 19.7 ± 1.8 | 17.6 ± 2.0 | 20.3 ± 2.9 |
| 4 | 27.3 ± 2.7 | 24.8 ± 1.7 | 19.4 ± 1.4 | 21.5 ± 1.0 | 29.2 ± 1.6 | 27.2 ± 1.1 |
| 7 | 27.1 ± 3.1 | 25.4 ± 1.1 | 23.8 ± 1.6 | 25.9 ± 1.9 | 31.6 ± 1.1 | 29.1 ± 1.0 |
| 12 | 28.0 ± 3.4 | 26.1 ± 1.2 | 25.0 ± 1.7 | 26.8 ± 1.0 | 32.8 ± 0.8 | 30.1 ± 1.4 |
| Expansion | 87% | 42% | 47% | 36% | 86% | 48% |
| Delta | | | | | | |

A = New increased absorbent capacity pledget design of the present invention
B = OB ® Digital Ultra Tampon

TABLE 3
Tampon Ejection Force Data from Samples of Increased
Absorbency Tampon Pledget of Present Invention

| Sample | Ejection Force |
|---|---|
| 1 | 17.86 |
| 2 | 24.02 |
| 3 | 23.11 |
| 4 | 25.17 |
| 5 | 22.23 |
| 6 | 20.28 |
| 7 | 18.24 |
| 8 | 22.11 |
| 9 | 21.02 |
| 10 | 15.36 |
| Average | 20.94 |
| Standard Deviation | 3.0455 |

TABLE 4
Comparison of Commercially Available High Absorbent
Capacity Tampons with Sample Increased Absorbency
Tampon Pledget of Present Invention

| | | | Newton | Embedded |
|---|---|---|---|---|
| | % | Plunge Pledget | Force | Length |
| | Embedded | Force N/mm | Total | (mm) |
| Gentle Glide Regular | 15.79 | 0.152 | 1.1236 | 7.4016 |
| Gentle Glide Super | 16.30 | 0.141 | 1.0720 | 7.5949 |
| Slimfits Regular | 15.66 | 0.073 | 0.5845 | 7.9538 |
| Walgreen's Regular | 9.19 | 0.114 | 0.5169 | 4.5496 |
| Pearl Regular | 4.97 | 0.192 | 0.4128 | 2.1539 |
| Pearl Super | 4.83 | 0.273 | 0.6107 | 2.2351 |
| Present Invention | 9.53 | 0.174 | 0.8896 | 5.1201 |

While particular embodiments of the present invention have been illustrated and described, it would be obvious to those skilled in the art that various other changes and modifications can be made without departing from the spirit and scope of the invention.

1. A tampon pledget comprising a compressed absorbent material with a density greater than about 0.20 g/cc and an absorbent capacity of 15 g to 18 g.

2. The tampon pledget of claim 1 wherein said compressed absorbent material has an expansion delta of at least 45%.

3. The tampon pledget of claim 1 wherein said compressed absorbent material has an expansion delta of at least 50%.

4. The tampon pledget of claim 1 wherein at least ⅔ of the length of the pledget has a density of at least 0.36 g/cc.

5. The tampon pledget of claim 1 further comprising an applicator; wherein said applicator includes a plunger and a barrel.

6. The tampon pledget of claim 5 wherein the pledget exerts a force in a range of about 0.050 N/mm to about 0.500 N/mm upon ejection of the pledget from the applicator by movement of the plunger.

7. The tampon pledget of claim 5 wherein said plunger exerts a force in a range of about 0.050 N/mm to about 0.300 N/mm upon ejection of the pledget from the applicator.

8. The tampon pledget of claim 5 having a radial expansion width at t=0 minutes in a range of about 12 mm to about 25 mm.

9. The tampon pledget of claim 8 having a radial expansion width at t=12 minutes, greater than about 25 mm.

10. The tampon pledget of claim 5 having a radial expansion width at t=0 minutes in a range of about 12 mm to about 20 mm.

11. The tampon pledget of claim 10 having a radial expansion width at t=12 minutes in a range of about 20 mm to about 40 mm.

12. The tampon pledget of claim 1 wherein at least ⅔ of the length of the pledget has a radial expansion width at t=12 minutes, greater than about 26 mm.

13. The tampon pledget of claim 1 wherein at least ⅔ of the length of the pledget has a radial expansion width at t=12 minutes, greater than about 28 mm.

14. The tampon pledget of claim 5 having an expansion rate at t=12 minutes, of at least 0.60 mm/minute.

15. The tampon pledget of claim 5 having an expansion rate at t=12 minutes in a range of about 0.60 mm/minute to about 0.65 mm/minute.

16. The tampon pledget of claim 5 having an expansion rate at t=12 minutes, of at least 0.65 mm/minute.

17. The tampon pledget of claim 1 wherein at least ⅔ of the length of the pledget has an expansion rate at t=12 minutes, of at least 0.65 mm/minute.

18. The tampon pledget of claim 5 having a radial expansion width at t=4 minutes in a range of about 18 mm to about 35 mm.

19. The tampon pledget of claim 5 having a radial expansion width at t=4 minutes in a range of about 19 mm to about 30 mm.

20. The tampon pledget of claim 1 wherein at least ⅔ of the length of the pledget has radial expansion width at t=4 minutes, greater than 25 mm.

21. The tampon pledget of claim 1 wherein at least ⅔ of the length of the pledget has a radial expansion width at t=4 minutes, greater than 27 mm.

22. The tampon pledget of claim 5 having a radial expansion width at t=7 minutes in a range of about 20 mm to about 35 mm.

23. The tampon pledget of claim 5 having a radial expansion width at t=7 minutes in a range of about 22 mm to about 33 mm.

24. The tampon pledget of claim 1 wherein at least ⅔ of the length of the pledget has a radial expansion width at t=7 minutes, greater than 25 mm.

25. The tampon pledget of claim 1 wherein at least ⅔ of the length of the pledget has a radial expansion width at t=7 minutes, greater than 27 mm.

26. The tampon pledget of claim 1 wherein the tampon pledget has a density in the range of about 0.20 g/cc to about 0.80 g/cc.
